(19) United States
(12) Patent Application Publication
(10) Pub. No.: US 2024/0279578 A1
(43) Pub. Date: Aug. 22, 2024

(54) CONTROLLING YEAST BLEND RATIOS, AND RELATED CONTROL SYSTEMS, APPARATUSES, AND METHODS

(71) Applicant: Novozymes A/S, Bagsvaerd (DK)

(72) Inventors: Kyle Rothanzl, Franklinton, NC (US); Patrick E. Williams, Kongens Lyngby (DK); Gregory Michael Ames, Copenhagen SV (DK); Scott Dickerson, Chapel Hill, NC (US); James Waterman, Aimes, IA (US); Laurie Duval, Ottawa (CA)

(73) Assignee: Novozymes A/S, Bagsvaerd (DK)

(21) Appl. No.: 18/569,099

(22) PCT Filed: Jun. 8, 2022
(86) PCT No.: PCT/US2022/032607

§ 371 Date: Dec. 11, 2023

Related U.S. Application Data
(60) Provisional application No. 63/209,665, filed on Jun. 11, 2021.
(60) Provisional application No. 63/253,458, filed on Oct. 7, 2021.
(60) Provisional application No. 63/270,941, filed on Oct. 22, 2021.
(60) Provisional application No. 63/312,505, filed on Feb. 22, 2022.

(51) Int. Cl. C12M 1/00; C12M 1/34; C12M 1/36; C12P 7/06

(52) U.S. Cl. CPC C12M 21/12; C12M 41/32; C12M 41/36; C12M 41/48; C12P 7/06

(57) ABSTRACT

A method of producing bioethanol in a bioethanol system and a control system for a bioethanol system is disclosed, the control system comprising a controller comprising one or more processors and an interface, wherein the one or more processors are configured to obtain a grain flour flow of grain flour; determine an input scheme based on the grain flour flow; and control one or more input devices of the bioethanol system according to the input scheme. The one or more processors may further be configured to sense one or more indicators associated with a fermentation (e.g., bioethanol) system, to determine a yeast blend ratio based at least partially on the one or more indicators, generate one or more control signals based on the determined yeast blend, and convey the one or more control signals to the fermentation system. Associated systems, apparatuses, and methods are also disclosed.

### TECHNICAL FIELD

The present disclosure relates to production of bioethanol and related control systems. In particular, a method of producing bioethanol in a bioethanol system is disclosed. Embodiments of the present disclosure generally relate to controlling yeast blend ratios, and more specifically to determining and/or dosing a yeast blend ratio based on one or more conditions. In particular, embodiments of the present disclosure relate to control systems for determining and/or dosing of yeast blend ratios, and related methods and apparatuses.

### BACKGROUND

Yeast and enzymes are ingredients used to produce products, such as bioethanol, which may be used to replace fossil fuels. Bioethanol may reduce CO2 emissions while allowing systems that conventionally operate on fossil fuels to continue operating with minimal alterations. Controlling the amount of yeast and/or enzyme used in a process of producing bioethanol effectively may improve the fermentation process, reduce the fermentation time, and/or reduce the amount of yeast and enzymes used. Developing systems for creating bioethanol with increased efficiency, repeatability, and reliability may reduce costs associated with the production of bioethanol making bioethanol a cost-effective replacement for fossil fuels.

### BRIEF SUMMARY

Accordingly, there is a need for methods and devices optimizing bioethanol yield and processes related to bioethanol production.

A control system for a bioethanol system or parts of a bioethanol system is disclosed, the control system comprising a controller comprising one or more processors and an interface. The one or more processors are configured to obtain, e.g. via the interface, a grain flour flow of grain flour; determine an input scheme based on the grain flour flow; and control, e.g. via the interface, one or more input devices of the bioethanol system according to the input scheme.

Further, a method for producing bioethanol in a bioethanol system is disclosed. The method comprises obtaining a grain flour flow of grain flour; determining an input scheme based on the grain flour flow; and controlling one or more input devices of the bioethanol system according to the input scheme.

The present disclosure allows optimization of slurry preparation and input, by controlling e.g., grain flour dosing, yeast dosing, enzyme dosing, to a tank (e.g., slurry, liquefaction, fermentation, etc.) of a bioethanol plant or system which in turn may lead to increased or optimized bioethanol yield.

It is an important advantage of the present disclosure that a more accurate and improved slurry preparation is provided which in turn allows for a more stable bioethanol production and reduces waste. In particular, the real-time monitoring and determination of the grain flour flow allows more precise control of the preparation process and/or liquefaction and/or fermentation.

Further, the use of in situ real-time online monitoring and control of the bioethanol system allows for a more stable input to the bioethanol system.

Embodiments of the present disclosure may include a yeast injection system. The system may include a first yeast container coupled to a mixing chamber through a first yeast input device. The system may further include a second yeast container coupled to the mixing chamber through a second yeast input device. The system may also include a controller configured to measure one or more process conditions associated with the mixing chamber and determine a yeast blend ratio based on the one or more process conditions. The controller may be configured to control the first yeast input device and the second yeast input device based on the yeast blend ratio.

Another embodiment of the present disclosure may include a bioethanol production system. The system may include a fermentation section and a controller configured to measure one or more process conditions in the bioethanol production system. The system may further include a yeast injection system coupled to the fermentation section. The yeast injection system may include a first yeast container coupled to the fermentation section through a first yeast input device. The yeast injection system may further include a second yeast container coupled to the fermentation section through a second yeast input device. The controller may be configured to determine a yeast blend ratio based on the one or more process conditions. The controller may further be configured to transmit a first yeast control parameter to the first yeast input device based on the yeast blend ratio. The controller may also be configured to transmit a second yeast control parameter to the second yeast input device based on the yeast blend ratio.

Another embodiment of the present disclosure may include a control system including one or more processors configured to communicatively couple with a bioethanol system. The one or more processors may be further configured to: sense one or more indicators associated with the fermentation system; determine a yeast blend ratio based at least partially on the one or more indicators; generate one or more control signals based on the determined yeast blend; and convey the one or more control signals to the fermentation system.

In yet another embodiment, a control system may include one or more processors configured to receive data including more indicators associated with a fermentation system; determine one or more yeast blend ratios based at least partially on the one or more indicators; generate one or more control signals based on the determined yeast blend; and convey the one or more control signals to the fermentation system.

### BRIEF DESCRIPTION OF THE DRAWINGS

While the specification concludes with claims particularly pointing out and distinctly claiming embodiments of the present disclosure, the advantages of embodiments of the disclosure may be more readily ascertained from the following description of embodiments of the disclosure when read in conjunction with the accompanying drawings in which:

FIG. 1 illustrates a schematic view of a bioethanol system in accordance with one or more embodiments of the present disclosure;

FIG. 2 illustrates a block diagram of a bioethanol system in accordance with one or more embodiments of the present disclosure;

FIG. 3 illustrates a block diagram of a yeast injection system in accordance with one or more embodiments of the present disclosure;

FIG. 4 illustrates a block diagram of a control system in accordance with one or more embodiments of the present disclosure;

FIG. 5 illustrates another block diagram of another control system, according to various embodiments of the present disclosure;

FIG. 6 illustrates another block diagram of yet another control system, according to various embodiments of the present disclosure; and

FIG. 7 is a flow chart of an exemplary method of producing bioethanol.

FIG. 8 shows corn mass measured in an ethanol plant by both the rotary feeder and by an embodiment of the control system of the present disclosure in which a weight meter (e.g., Ronan) is used for accurate, real-time measurement of corn mass, illustrating the error of the rotary feeder measurement, which error was predominant in the industry before the control system of the present disclosure.

FIG. 9 illustrates the high variability of enzyme dosing (alpha-amylase (AA)) when grain mass is highly variable and alpha-amylase is added to the slurry at a constant rate (left) and reduced variability of enzyme dosing (AA) when an embodiment of the control system of the present disclosure accurately measures flour mass and adjusts the enzyme pump in real time so that the enzyme added is proportional to the ground flour to maintain a stable w/w dosage of enzyme (right).

FIG. 10A and FIG. 10B show the distribution of corn grind rates at two different ethanol plants. The top half of each panel show the distribution of grind rates before implementation of grind control of grain using an embodiment of the control system of the present disclosure, while the bottom half show the grind rates after. By comparing standard deviations we can see that variability was reduced by 39% for the ethanol plant in FIG. 10A, and by 29% for the ethanol plant in FIG. 10B.

FIG. 11 shows the distribution of alpha amylase (AA) dosing rates for a dry-grind ethanol plant. The top half shows the AA dosing rates before implementation of the enzyme dosing scheme using an embodiment of the control system of the present disclosure, while the bottom half show the AA dosing rates after. By comparing standard deviations we can see that variability was reduced by over 33% upon implementation of the control system of the present disclosure.

FIG. 12 illustrates the relationship between alpha amylase (AA) dose for two different AA molecules at different temperatures. The line describing the relationship of viscosity decreasing with increased AA dose explains more than 80% of the observed data.

FIG. 13 illustrates dextrinization (Green) and Solubilization (Black) of starch in corn mash at varying doses of an alpha amylase at standard industry conditions for temperature and solids.

### DETAILED DESCRIPTION

The illustrations presented herein are not meant to be actual views of any particular apparatus or system or component thereof, but are merely idealized representations employed to describe illustrative embodiments. The drawings are not necessarily to scale.

As used herein, the term “substantially” in reference to a given parameter means and includes to a degree that one skilled in the art would understand that the given parameter, property, or condition is met with a small degree of variance, such as within acceptable manufacturing tolerances. For example, a parameter that is substantially met may be at least about 90% met, at least about 95% met, at least about 99% met, or even at least about 100% met.

As used herein, relational terms, such as “first,” “second,” “top,” “bottom,” etc., are generally used for clarity and convenience in understanding the disclosure and accompanying drawings and do not connote or depend on any specific preference, orientation, or order, except where the context clearly indicates otherwise.

As used herein, the term “and/or” means and includes any and all combinations of one or more of the associated listed items.

As used herein, the terms “vertical” and “lateral” refer to the orientations as depicted in the figures.

A control system for a bioethanol system or parts of a bioethanol system is disclosed. The control system comprises a controller comprising one or more processors and an interface.

The control system optionally comprises a sensor system. The sensor system comprises one or more sensors connected to controller(s) of the control system for provision of sensor data to the controller. The control system may be a distributed control system. In other words, the control system may comprise a plurality of controllers, each controller implementing one or more control schemes to control the bioethanol system or parts thereof.

The one or more processors of the controller are configured to obtain, such as one or more of determine, measure, receive, and retrieve, a grain flour flow of grain flour and/or a grain flow, e.g. at a conveyor of the bioethanol system. The grain flour flow also denoted GFF and/or the grain flow GF may be obtained based on grain flour data/sensor data from one or more sensors arranged in the bioethanol system, such as at a conveyer or an output of a milling device/grinder. In other words, the grain flour flow and/or the grain flow may be obtained as a directly measured mass flow rate of grain flour.

The one or more processors of the controller are configured to determine an input scheme based on the grain flour flow. The input scheme may comprise and define control parameters for one or more input devices in the bioethanol system, e.g. for one or more pump devices, one or more grinders, one or more conveyors or other device(s) operating as input devices in the bioethanol system.

The one or more processors of the controller are configured to control one or more input devices of the bioethanol system according to the input scheme. For example, to control one or more input devices of the bioethanol system may comprise to output or transmit, e.g. via the interface, one or more control parameters also denoted input control parameters ICPs to input device(s) of the bioethanol system.

The input scheme may comprise an enzyme dosing scheme. The enzyme dosing scheme is indicative of and defines enzyme control parameters also denoted ECP's for controlling enzyme dosing in the bioethanol system.

In one or more example controllers, to determine an input scheme comprises to determine an enzyme dosing scheme, the enzyme dosing scheme comprising a first enzyme flowrate or first enzyme amount (first enzyme control parameter) for a first enzyme, and wherein to control one or more input devices comprises to control a first enzyme input device according to the enzyme dosing scheme, such as a first enzyme flowrate or first enzyme amount. In other words, the enzyme dosing scheme may comprise a first enzyme control parameter ECP_1, such as a first enzyme flowrate and/or a first enzyme amount or indicative thereof.

The first enzyme may be a first enzyme or a first enzyme composition.

In one or more example controllers, the first enzyme is selected from alpha-amylase, endoglucanase, glucoamylase, phospholipase, phytase, protease, pullulanase, and xylanase or is a first enzyme composition comprising one or more of alpha-amylase, endoglucanase, glucoamylase, phospholipase, phytase, protease, pullulanase, and xylanase.

In one or more example controllers, to control an input device comprises to control the first enzyme input device according to the first enzyme control parameter, such as first enzyme flowrate and/or the first enzyme amount. In other words, the input scheme may comprise an enzyme dosing scheme comprising a first enzyme control parameter, and the controller may be configured to output or transmit a first enzyme control parameter ECP_1 indicative of or being the first enzyme flowrate and/or the first enzyme amount, e.g. to the first enzyme input device, such as a dosing pump or valve.

In one or more example controllers, the enzyme dosing scheme comprises a second enzyme flowrate or second enzyme amount (second enzyme control parameter) for a second enzyme, and wherein to control an input device comprises to control a second enzyme input device according to the enzyme dosing scheme, such as the second enzyme flowrate and/or second enzyme amount. In other words, the enzyme dosing scheme may comprise a second enzyme control parameter ECP_2, such as a second enzyme flowrate and/or a second enzyme amount or indicative thereof.

The second enzyme may be a second enzyme or a second enzyme composition. The second enzyme composition may comprise the first enzyme and/or one or more additional enzymes.

In one or more example controllers, the second enzyme is selected from alpha-amylase, endoglucanase, glucoamylase, phospholipase, phytase, protease, pullulanase, and xylanase or is a second enzyme composition comprising one or more of alpha-amylase, endoglucanase, glucoamylase, phospholipase, phytase, protease, pullulanase, and xylanase. The second enzyme may be different from the first enzyme. In other words, the controller may be configured to determine and control input of a plurality of enzymes or enzyme compositions, thereby allowing tailoring the input on enzymes to the input of grain flour.

In one or more example controllers, to control an input device comprises to control the second enzyme input device according to the second enzyme control parameter, such as the second enzyme flowrate and/or the second enzyme amount. In other words, the input scheme may comprise an enzyme dosing scheme comprising a second enzyme control parameter, and the controller may be configured to output or transmit a second enzyme control parameter ECP_2 indicative of or being the second enzyme flowrate and/or the second enzyme amount, e.g. to the second enzyme input device, such as a dosing pump or valve.

The second enzyme may operate on a by-product of the first enzymatic process carried out by the first enzyme.

The enzyme dosing scheme may define a rate or a difference between different enzyme flowrates. For example, the enzyme dosing scheme may comprise a first ratio indicative of ratio between a first enzyme flowrate and a second enzyme flowrate. In other words, to control an input device may comprise to control a ratio between input of the first enzyme and input of the second enzyme.

In one or more example controllers, the enzyme dosing scheme comprises a third enzyme flowrate or third enzyme amount (third enzyme control parameter) for a third enzyme, and wherein to control an input device comprises to control a third enzyme input device according to the enzyme dosing scheme, such as the third enzyme flowrate and/or third enzyme amount. In other words, the enzyme dosing scheme may comprise a third enzyme control parameter ECP_3, such as a third enzyme flowrate and/or a third enzyme amount or indicative thereof.

The third enzyme may be a third enzyme or a third enzyme composition. The third enzyme composition may comprise the first enzyme, the second enzyme, and/or one or more additional enzymes.

In one or more example controllers, the third enzyme is selected from alpha-amylase, endoglucanase, glucoamylase, phospholipase, phytase, protease, pullulanase, and xylanase or is a third enzyme composition comprising one or more of alpha-amylase, endoglucanase, glucoamylase, phospholipase, phytase, protease, pullulanase, and xylanase. The third enzyme may be different from the first enzyme and/or different from the second enzyme.

In one or more example controllers, to control an input device comprises to control the third enzyme input device according to the third enzyme control parameter, such as the third enzyme flowrate and/or the third enzyme amount. In other words, the input scheme may comprise an enzyme dosing scheme comprising a third enzyme control parameter, and the controller may be configured to output or transmit a third enzyme control parameter ECP_3 indicative of or being the third enzyme flowrate and/or the third enzyme amount, e.g. to the third enzyme input device, such as a dosing pump or valve.

The third enzyme may operate on a by-product of one or both of the first enzymatic process carried out by the first enzyme and the second enzymatic process carried out by the second enzyme.

In one or more example controllers, the enzyme dosing scheme may comprise a ratio indicative of ratio between a first enzyme flowrate and a third enzyme flowrate. In other words, to control an input device may comprise to control a ratio between input of the first enzyme and input of the third enzyme.

In one or more example controllers, the enzyme dosing scheme may comprise a ratio indicative of ratio between a second enzyme flowrate and a third enzyme flowrate. In other words, to control an input device may comprise to control a ratio between input of the second enzyme and input of the third enzyme.

In one or more example controllers, the enzyme dosing scheme may comprise a ratio indicative of ratio between a combined enzyme flowrate, e.g. flowrate of first enzyme and second enzyme, and a third enzyme flowrate. In other words, to control an input device may comprise to control a ratio between input of the combination of first enzyme/second enzyme and input of the third enzyme.

In one or more example controllers, the enzyme dosing scheme comprises a fourth enzyme flowrate or fourth enzyme amount (fourth enzyme control parameter) for a fourth enzyme, and wherein to control an input device comprises to control a fourth enzyme input device according to the enzyme dosing scheme, such as the fourth enzyme flowrate and/or fourth enzyme amount. In other words, the enzyme dosing scheme may comprise a fourth enzyme control parameter ECP_4, such as a fourth enzyme flowrate and/or a fourth enzyme amount or indicative thereof.

The fourth enzyme may be a fourth enzyme or a fourth enzyme composition. The fourth enzyme composition may comprise the first enzyme, the second enzyme, the third enzyme, and/or one or more additional enzymes.

In one or more example controllers, the fourth enzyme is selected from alpha-amylase, endoglucanase, glucoamylase, phospholipase, phytase, protease, pullulanase, and xylanase or is a fourth enzyme composition comprising one or more of alpha-amylase, endoglucanase, glucoamylase, phospholipase, phytase, protease, pullulanase, and xylanase. The fourth enzyme may be different from the first enzyme and/or different from the second enzyme and/or different from the third enzyme.

The fourth enzyme may operate on a by-product of one or more of the first enzymatic process carried out by the first enzyme, the second enzymatic process carried out by the second enzyme, and/or the third enzymatic process carried out by the third enzyme.

In one or more example controllers, to control an input device comprises to control the fourth enzyme input device according to the fourth enzyme control parameter, such as the fourth enzyme flowrate and/or the fourth enzyme amount. In other words, the input scheme may comprise an enzyme dosing scheme comprising a fourth enzyme control parameter, and the controller may be configured to output or transmit a fourth enzyme control parameter ECP_4 indicative of or being the fourth enzyme flowrate and/or the fourth enzyme amount, e.g. to the fourth enzyme input device, such as a dosing pump or valve.

In other words, to determine an input scheme/enzyme dosing scheme may comprise determining one or more enzyme control parameters optionally including one or more of first enzyme control parameter, second enzyme control parameter, third enzyme control parameter, and fourth enzyme control parameter based on the grain flour flow.

In one or more example controllers, to determine an input scheme comprises to determine a yeast dosing scheme, the yeast dosing scheme comprising a first yeast flowrate or a first yeast amount of a first yeast (first yeast control parameter), and wherein to control one or more input devices comprises to control a first yeast input device according to the yeast dosing scheme, such as the first yeast flowrate or first yeast amount. In other words, the yeast dosing scheme may comprise a first yeast control parameter YCP_1, such as a first yeast flowrate and/or a first yeast amount or indicative thereof. The first yeast may be a first yeast or a first yeast composition. The first yeast may be formulated as a cream yeast or a dry yeast. A cream yeast may allow precise yeast dosing in an input device.

In one or more example controllers, the first yeast is selected from Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp., or is a first yeast composition comprising one or more of Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp.

In one or more example controllers, to control an input device comprises to control the first yeast input device according to the first yeast control parameter, such as first yeast flowrate and/or the first yeast amount. In other words, the input scheme may comprise a yeast dosing scheme comprising a first yeast control parameter, and the controller may be configured to output or transmit a first yeast control parameter YCP_1 indicative of or being the first yeast flowrate and/or the first yeast amount, e.g. to the first yeast input device, such as a dosing pump or valve.

In one or more example controllers, the yeast dosing scheme comprises a second yeast flowrate or a second yeast amount of a second yeast (second yeast control parameter), and wherein to control an input device comprises to control a second yeast input device according to the second yeast flowrate.

In one or more example controllers, the yeast dosing scheme comprises a second yeast flowrate or a second yeast amount of a second yeast (second yeast control parameter), and wherein to control one or more input devices comprises to control a second yeast input device according to the yeast dosing scheme, such as the second yeast flowrate or second yeast amount. In other words, the yeast dosing scheme may comprise a second yeast control parameter YCP_2, such as a second yeast flowrate and/or a second yeast amount or indicative thereof. The second yeast may be a second yeast or a second yeast composition. The second yeast may be formulated as a cream yeast or a dry yeast. A cream yeast may allow precise yeast dosing in an input device.

In one or more example controllers, the second yeast is selected from Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp., or is a first yeast composition comprising one or more of Saccharomyces, Rhodotorula, Schizosaccharomyces, Kluyveromyces, Pichia, Hansenula, Rhodosporidium, Candida, Yarrowia, Lipomyces, Cryptococcus,and Dekkera sp.

In one or more example controllers, to control an input device comprises to control the second yeast input device according to the second yeast control parameter, such as second yeast flowrate and/or the second yeast amount. In other words, the input scheme may comprise a yeast dosing scheme comprising a second yeast control parameter, and the controller may be configured to output or transmit a second yeast control parameter YCP_2 indicative of or being the second yeast flowrate and/or the second yeast amount, e.g. to the second yeast input device, such as a dosing pump or valve.

In other words, to determine an input scheme/yeast dosing scheme may comprise determining one or more yeast control parameters optionally including a first yeast control parameter and/or a second yeast control parameter, based on the grain flour flow.

In one or more example controllers, to determine an input scheme comprises to determine a feed rate and/or a feed scheme, e.g. of grain, and wherein to control one or more input devices optionally comprises to control a feeder input device according to the feed rate/feed scheme. The feed scheme optionally comprises a first feed rate or first feed amount for a first feeder input device, e.g. for feeding grain or other raw material to the bioethanol system. To control one or more input devices optionally comprises to control a first feeder input device according to the feed rate/feed scheme, such as a first feed rate or first feed amount. In other words, the feed scheme may comprise a first feed control parameter FCP_1, such as a first feed rate and/or a first feed amount or indicative thereof.

In one or more example controllers, to control an input device comprises to control the first feeder input device according to the first feed control parameter, such as (first) feed rate and/or the (first) feed amount. In other words, the input scheme may comprise a feed rate and/or a feed scheme comprising a first feed control parameter, and the controller may be configured to output or transmit a first feed control parameter FCP_1 indicative of or being the first feed rate and/or the first feed amount, e.g. to the first feeder input device. The first feeder input device may be a rotary valve.

In one or more example controllers, to determine a feed scheme comprises to determine a second feed rate or second feed amount for a second feeder input device, e.g. for feeding grain or other raw material to the bioethanol system, and wherein to control one or more input devices optionally comprises to control a second feeder input device according to the feed scheme, such as the second feed rate or second feed amount for a second feeder input device, e.g. for feeding grain or other raw material to the bioethanol system. In other words, the feed scheme may comprise a second feed control parameter FCP_2, such as a second feed rate and/or a second feed amount or indicative thereof.

In one or more example controllers, to control an input device comprises to control the second feeder input device according to the second feed control parameter, such as second feed rate and/or the second feed amount. In other words, the input scheme may comprise a feed rate and/or a feed scheme comprising a second feed control parameter, and the controller may be configured to output or transmit a second feed control parameter FCP_2 indicative of or being the second feed rate and/or the second feed amount, e.g. to the second feeder input device. The second feeder input device may be a rotary valve.

In one or more example controllers, to determine a feed scheme comprises to determine a third feed rate or third feed amount for a third feeder input device, e.g. for feeding grain or other raw material to the bioethanol system, and wherein to control one or more input devices optionally comprises to control a third feeder input device according to the feed scheme, such as the third feed rate or third feed amount for a third feeder input device, e.g. for feeding grain or other raw material to the bioethanol system. In other words, the feed scheme may comprise a third feed control parameter FCP_3, such as a third feed rate and/or a third feed amount or indicative thereof.

In one or more example controllers, to control an input device comprises to control the third feeder input device according to the third feed control parameter, such as third feed rate and/or the third feed amount. In other words, the input scheme may comprise a feed rate and/or a feed scheme comprising a third feed control parameter, and the controller may be configured to output or transmit a third feed control parameter FCP_3 indicative of or being the third feed rate and/or the third feed amount, e.g. to the third feeder input device. The third feeder input device may be a rotary valve.

The control system may comprise one or more sensors including a first sensor and/or a second sensor. The sensor(s) provide sensor data for the controller(s), e.g. for determining the input scheme based on the sensor data.

In one or more example control systems, the control system comprises a weight meter (first sensor or first sensor device) for provision of weight data of grain flour and wherein to obtain a grain flour flow comprises to determine the grain flour flow based on the weight data. The weight meter may be a Ronan density meter.

In one or more example control systems, the control system comprises a speedometer (second sensor or second sensor device) for provision of speed data, e.g. of a conveyor such as a belt conveyor, and wherein to obtain a grain flour flow comprises to determine the grain flour flow based on the speed data.

In a typical single stream, dry-grind ethanol plant using only corn as feedstock, weight meters (e.g., Ronan device) alone can be used to make reasonably accurate enzyme dosing decisions. This, however, assumes that the starch content of the incoming corn is not highly variable and so the mass of corn going into the process is proportional to the mass of flour going into the process. Often this is not the case and particularly in cases where plants are using mixed feedstocks going into the mill. In such cases in order to accurately determine the optimal enzyme dosing scheme (and which classes of enzymes to dose) a more accurate in-line determination of the amounts of starch, protein, fiber, fat, and water is needed. Therefore, in one or more example control systems, the control system comprises a spectrometer (third sensor or third sensor device) for provision of spectrometer data of grain flour and wherein to obtain a grain flour flow comprises to determine a first component flow or a first component amount of a first component of the grain flour based on the spectrometer data and the grain flour flow. The first component may be one of a starch, a protein, a fiber, a fat, or moisture content. Accordingly, the first component flow may be a starch flow or amount of starch, a protein flow or amount of protein, a fiber flow or amount of fiber, a fat flow, or amount of fat, and moisture content or amount of moisture. The third sensor/sensor device, such as the spectrometer, may be arranged at a (belt) conveyor and/or at an output of a grinder e.g. of a preparation section of the bioethanol system. The spectrometer may comprise one or more near infrared (NIR) sensors. The NIR spectrometer may be a Zeiss NIR spectrometer. The NIR spectrometer may be a BUCHI NIR spectrometer. The NIR spectrometer may be a PERTEN NIR spectrometer. The NIR spectrometer may be a METROHM NIR spectrometer. The spectrometer may be an FTIR spectrometer. Using one or more spectrometer sensors (e.g., in-line NIR) to measure the crude nutrient content of the incoming feedstock (% starch, % fat, % protein, % fiber, and % water), and by coupling this flour composition data from the NIR with flour mass data from the weight meter (e.g., Ronan device) enables calculating the mass of each component (starch/fat/protein/fiber) going into the process. This will in turn allow more accurate dosing of enzymes into the process, e.g, alpha-amylase dosing based on the mass of starch, protease dosing based on mass of protein, xylanase dosing based on the mass of fiber etc. This NIR technology has particular value in the case of mixed feedstocks, where there may be a high degree of variation of the above component contents. The addition of an in-line NIR to the flour feed allows the measurement of e.g., incoming starch regardless of which feedstock it was derived from, or variability in the mixing ratio of multiple feedstocks. The feedstock may in one embodiment be selected from the group comprising corn, wheat, barley, rye, milo, sago, cassava, tapioca, sorghum, oat, rice, peas, beans, beats, sweet potatoes, other sources of starch, such as e.g., starch waste by-products, or mixtures thereof. In one embodiment the mixed feedstock may comprise corn and milo. In another embodiment the mixed feedstock may comprise corn and wheat.

In one or more example controllers, to determine a first enzyme flowrate or a first enzyme amount for a first enzyme is based on the first component flow. In one or more example controllers, the first enzyme is alpha-amylase and the first component is starch.

In one or more example controllers, to determine a second enzyme flowrate or a second enzyme amount for a second enzyme is based on the second component flow. In one or more example controllers, the second enzyme is protease and the second component is protein.

In one or more example controllers, to determine a third enzyme flowrate or a third enzyme amount for a third enzyme is based on the third component flow. In one or more example controllers, the third enzyme is a xylanase and the third component is fiber.

In one or more example controllers, to determine a fourth enzyme flowrate or a fourth enzyme amount for a fourth enzyme is based on the fourth component flow. In one or more example controllers, the fourth enzyme is protease and the fourth component is fat. In one or more example controllers, the fourth enzyme is lipase (e.g., phospholipase) and the fourth component is fat.

In one or more example controllers, to obtain a grain flour flow comprises to determine a second component flow or a second component amount of a second component of the grain flour based on the spectrometer data and the grain flour flow. The second component may be one of a protein, a fiber, a fat, and a moisture content. Accordingly, the second component flow may be a protein flow, a fiber flow, a fat flow, or a moisture content flow. In one or more example controllers, the second enzyme is protease and the second component is protein.

In one or more example controllers, to obtain a grain flour flow comprises to determine a third component flow or a third component amount of a third component of the grain flour based on the spectrometer data and the grain flour flow. The third component may be fiber. Accordingly, the third component flow may be a fiber flow. In one or more example controllers, the third enzyme is xylanase and the third component is fiber. The fiber may comprise cellulose or hemicellulose.

In one or more example controllers, to obtain a grain flour flow comprises to determine a fourth component flow or a fourth component amount of the grain flour based on the spectrometer data and the grain flour flow. The fourth component may be fat. Accordingly, the fourth component flow may be a fat flow. In one or more example controllers, the fourth enzyme is lipase (e.g., phospholipase) and the fourth component is fat.

A component flow may be a combined component flow for a plurality of components. For example, a component flow, such as the first component flow and/or the second component flow and/or the third component flow and/or the fourth component flow may be a combined component flow, e.g. a component flow of starch and protein, a component flow of starch and fiber, a component flow of protein and fiber, a component flow of starch and fat, a component flow of protein and fat, a component flow of fiber and fat, a component flow of starch, protein and fiber, a component flow of starch, protein and fat, a component flow of protein, fiber and fat, a component flow of starch, fiber and fat, or a component flow of protein, fiber, starch and fat.

In one or more example controllers, to determine a second enzyme flowrate for a second enzyme is based on the second component flow and/or one or more other component flows, such as the first component flow and/or the third component flow of a third component and/or the fourth component flow of the fourth component.

In one or more example controllers, to determine a first enzyme flowrate for a first enzyme is based on the first component flow and/or one or more other components flows, such as the second component flow and/or the third component flow of the third component and/or the fourth component flow of the fourth component.

In one or more example control systems, the control system comprises a spectrometer (fourth sensor or fourth sensor device) for provision of spectrometer data of a liquefact/liquefied mash and wherein to obtain a grain flour flow comprises to determine the first component flow or the first component amount of the first component of the grain flour based on the spectrometer data from the third sensor or third sensor device and/or fourth sensor or fourth sensor device and the grain flour flow. The fourth sensor/sensor device, such as the spectrometer, may be arranged at an output of the liquefaction tank of the liquefaction section of the bioethanol system.

In one or more example controllers, the spectrometer data of the liquefact/liquefied mash provided by the fourth sensor/sensor device can be used to determine the effectiveness of the first enzyme on the first component flow or the first component amount of the first component of the grain flour, and to determine whether the first enzyme flow rate needs to be optimized based on changes in grain flour flow. For example, if the first enzyme is alpha-amylase and the first component flow is starch, the fourth sensor/sensor device can detect sugar profiles (e.g., DP1, DP2, DP3, DP4, etc.) that can be used by the control system to determine whether adjustments in alpha-amylase dosing need to be made based on the amount of starch remaining post-liquefaction as detected by the fourth sensor or fourth sensor device relative to the amount of starch present in the grain flour flow measured by the third sensor or third sensor device.

In one or more example controllers, the spectrometer data of the liquefact/liquefied mash provided by the fourth sensor/sensor device can be used to determine the effectiveness of the second enzyme on the second component flow or the second component amount of the grain flour, and to determine whether the second enzyme flow rate needs to be optimized based on changes in gain flour flow. For example, if the second enzyme is protease and the second component flow is protein, the fourth sensor/sensor device can detect protein (e.g., soluble protein) that can be used by the control system to determine whether adjustments in protease dosing need to be made based on the amount of soluble protein present post-liquefaction as detected by the fourth sensor or fourth sensor device relative to the amount of protein present in the grain flour flow measured by the third sensor or third sensor device.

The spectrometer (fourth sensor/fourth sensor device) may comprise one or more near infrared (NIR) sensors. The NIR spectrometer may be a PERTEN NIR spectrometer. The NIR spectrometer may be a METROHM NIR spectrometer. The spectrometer (fourth sensor/fourth sensor device) may comprise one or more mid infrared (MIR) sensors. The MIR spectrometer may be a KEIT MIR spectrometer. The spectrometer may be an FTIR spectrometer.

The present disclosure relates to a method of producing bioethanol in a bioethanol system, wherein the method comprises obtaining a grain flour flow of grain flour; determining an input scheme based on the grain flour flow; and controlling one or more input devices of the bioethanol system according to the input scheme.

It is noted that descriptions and features of controller and controller functionality also applies to methods and vice versa.

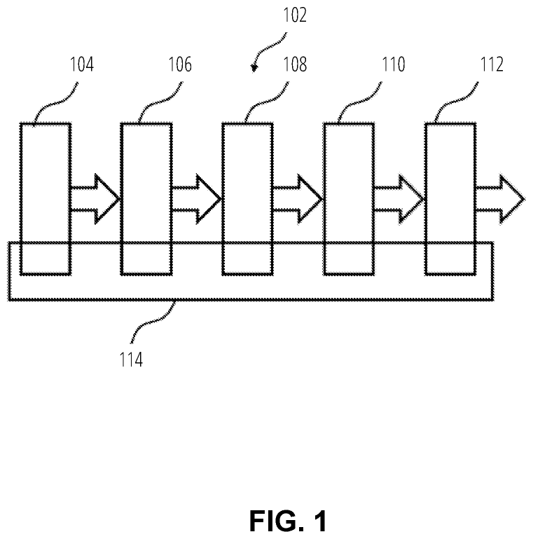

FIG. 1 shows an exemplary bioethanol system or bioethanol plant implementing a control system according to the present disclosure. A bioethanol system 102 comprises a preparation section 104 where grain is milled to flour and combined with water (and optionally enzymes) to form a slurry, a liquefaction section 106 where high temperature is used in combination with enzymes, such as alpha-amylase, to hydrolyze the starch present in the slurry to a dextrins, a fermentation section 108 where the dextrins are saccharified via enzymes, such as glucoamylase, to fermentable sugars and the fermentable sugars are used by a fermenting organism (e.g., yeast) to produce ethanol, an ethanol production section 110, and a separation section 112. A control system 114 controls devices and operations in one or more of the sections 104, 106, 108, 110, 112. The control system 114 may be a distributed controls system with a central controller and one or more section controllers distributed in respective sections 104, 106, 108, 110, 112.

The control system 114 for the bioethanol system 102 or respective sections 104, 106, 108, 110, 112 may include a controller including one or more processors and an interface. The control system 114 may include a sensor system. The sensor system may include one or more sensors connected to controller(s) of the control system 114 for provision of sensor data to the controller. The control system 114 may be a distributed control system. In other words, the control system 114 may include a plurality of controllers, each controller implementing one or more control schemes to control the bioethanol system 102, respective sections 104, 106, 108, 110, 112, or parts thereof.

The control system 114 may include one or more sensors. The sensor(s) provide sensor data for the controller(s), which may process the sensor data and perform calculations and/or make determinations based on the sensor data. The control system 114 may include a weight meter, such as a density meter, for measuring weight data of grain flour, slurries, and/or other fluids throughout the bioethanol system 102. For example, a grain flour flow may be determined based on the grain flour flow based on the weight data. The control system 114 may further include a speedometer or flowmeter for measuring speed data, such as the speed of a conveyor or flow velocity. For example, the grain flour flow may be determined based on the speed data.

The control system 114 may include a spectrometer for measuring spectrometer data of grain flour and/or the mixtures within the respective sections 104, 106, 108, 110, 112 of the bioethanol system 102. The spectrometer may be used to determine a component flow, a component amount, an organic acid content, etc., of components of the grain flour and/or the mixtures within the respective sections 104, 106, 108, 110, 112 of the bioethanol system 102 based on the spectrometer data. For example, the components may be one of a starch, a protein, a fiber, a fat, or moisture content. Accordingly, the component flow may be a starch flow or amount of starch, a protein flow or amount of protein, a fiber flow or amount of fiber, a fat flow, or amount of fat, and moisture content or amount of moisture. The spectrometer, may be arranged at a (belt) conveyor, at an output of a grinder of the preparation section 104, at an output of the preparation section 104, at an output of the liquefaction section 106, at an output of the fermentation section 108, and/or at an output of the production section 110. The spectrometer may be a near infrared (NIR) spectrometer, mid-infrared (MIR) spectrometer, or a Fourier transform infrared (FTIR) spectrometer.

The one or more processors of the controller are configured to determine, measure, receive, and retrieve, process conditions, such as a grain flour flow, a grain flow, fermentation time, fermentation solids present, process temperatures, fermentation products, fermentation by-products, such as glycerol and organic acids (e.g., acetic acid, lactic acid, etc.) present, in different locations throughout the bioethanol system 102, based on sensor readings throughout the bioethanol system 102. For example, the one or more processors of the control system 114 may determine process conditions before a mill or grinding element in the preparation section 104, after the mill or grinding element in the preparation section 104, on a conveyor of the bioethanol system 102, entering the liquefaction section 106, within the liquefaction section 106, leaving the liquefaction section 106, entering the fermentation section 108, within the fermentation section 108, leaving the fermentation section 108, entering the production section 110, within the production section 110, leaving the production section 110, entering the separation section 112, within the separation section 112, leaving the separation section 112, etc.

The grain flour flow also denoted GFF and/or the grain flow GF may be obtained based on grain flour data/sensor data from one or more sensors arranged in the bioethanol system, such as at the conveyer or an output of the mill or grinding element in the preparation section 104. In other words, the grain flour flow and/or the grain flow may be obtained as a directly measured mass flow rate of grain flour.

The one or more processors of the controller are configured to determine an input scheme based on the grain flour flow. The input scheme may comprise and define control parameters for one or more input devices in the bioethanol system 102, e.g. for one or more pump devices, one or more grinders, one or more conveyors or other device(s) operating as input devices in the bioethanol system 102.

The one or more processors of the controller are configured to control one or more input devices of the bioethanol system 102 according to the input scheme. For example, controlling one or more input devices of the bioethanol system 102 may include outputting or transmitting one or more control parameters also denoted input control parameters ICPs to the input device(s) of the bioethanol system 102, such as through a direct connection, the interface described above, and/or a network connection between individual controllers.

One or more of the controllers may determine an input scheme to determine an enzyme dosing scheme. The enzyme dosing scheme may control the amount of different enzymes introduced in the liquefaction section 106 and/or the preparation section 104. The enzyme dosing scheme may include a first enzyme flowrate or first enzyme amount (first enzyme control parameter or ECP_1) for a first enzyme. The one or more controllers may then control a first enzyme input device, such as a dosing pump or dosing valve, according to the enzyme dosing scheme, such as controlling the first enzyme input device to the first enzyme flowrate or first enzyme amount.

The first enzyme may be a first enzyme or a first enzyme composition. For example, the first enzyme may be alpha-amylase, endoglucanase, glucoamylase, phospholipase, phytase, protease, pullulanase, and xylanase or is a first enzyme composition comprising one or more of alpha-amylase, endoglucanase, glucoamylase, phospholipase, phytase, protease, pullulanase, or xylanase.

The enzyme dosing scheme may further include a second enzyme flowrate or second enzyme amount (e.g., second enzyme control parameter or ECP_2) for a second enzyme. The one or more controllers may control a second enzyme input device according to the enzyme dosing scheme, such as the second enzyme flowrate and/or second enzyme amount. The second enzyme may be alpha-amylase, endoglucanase, glucoamylase, phospholipase, phytase, protease, pullulanase, and xylanase or is a second enzyme composition comprising one or more of alpha-amylase, endoglucanase, glucoamylase, phospholipase, phytase, protease, pullulanase, and xylanase. The second enzyme may be different from the first enzyme. In other words, the controller may be configured to determine and control input of a plurality of enzymes or enzyme compositions, thereby allowing tailoring the input on enzymes to the input of grain flour. The enzyme dosing scheme may include additional enzyme control parameters, such as third enzyme control parameters (ECP_3) and fourth enzyme control parameters (ECP_4) for controlling the input of additional enzymes, such as a third enzyme and a fourth enzyme.

The input scheme may also include a yeast dosing scheme. The yeast dosing scheme may define yeast control parameters for controlling yeast dosing in the bioethanol system 102, such as yeast dosing in the fermentation section 108 of the bioethanol system 102.

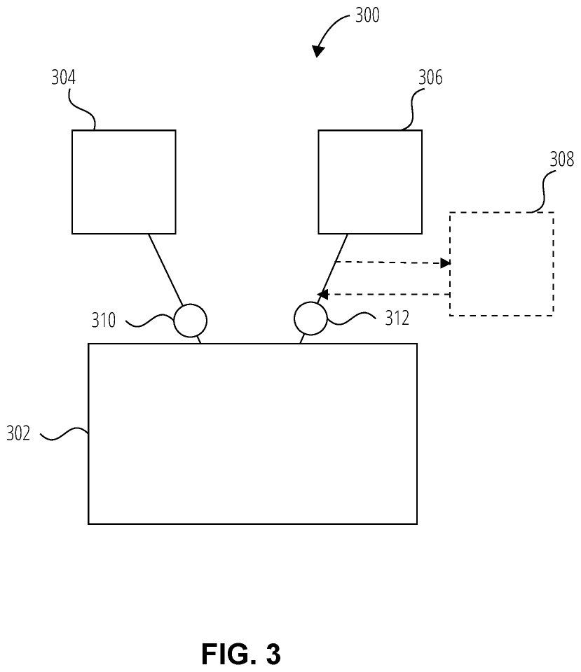

The yeast dosing scheme may include a first yeast flowrate or a first yeast amount of a first yeast (e.g., a first yeast control parameter or YCP_1). The controller may control a first yeast input device (e.g., yeast input device 310 (FIG. 3)), such as a dosing pump or valve, according to the yeast dosing scheme. For example, the first yeast input device may be controlled to the first yeast flowrate or first yeast amount. The first yeast may be formulated as a cream yeast or a dry yeast. A cream yeast may allow precise yeast dosing in an input device. The first yeast may be one or more of Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp., or a composition thereof.

The yeast dosing scheme may include a second yeast flowrate or a second yeast amount of a second yeast (e.g., second yeast control parameter or YCP_2). The controller may control a second yeast input device (e.g., yeast input device 312 (FIG. 3)), such as a dosing pump or valve, according to the second yeast flowrate. The second yeast may be a second yeast or a second yeast composition. The second yeast may be formulated as a cream yeast or a dry yeast. A cream yeast may allow precise yeast dosing in an input device. The second yeast may be one or more of Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp., or a composition thereof.

Determining an input scheme or yeast dosing scheme may include determining one or more yeast control parameters based on the grain flour flow. The yeast dosing scheme may further be determined based on a yeast blend ratio. The yeast blend ratio may be determined based on one or more mathematical, statistical, or machine learning models that predict fermentation product yield (e.g., ethanol) from a controlled fermentation process based on one or more process conditions (also referred to herein as “operating conditions”). The process conditions may be one or more of known or unknown (such as in the case of a machine learning model) indications of fermentation performance. Non-limiting examples of the one or more process conditions include: fermentation time, fermentation solids present, process temperatures, fermentation products, fermentation by-products, such as glycerol and organic acids (e.g., acetic acid, lactic acid) present, etc. Values for the one or more process conditions may be captured by, or determined based on, sensor readings in one or more of the preparation section 104, liquefaction section 106, fermentation section 108, or ethanol production section 110 or lab testing on samples taken from one or more of the preparation section 104, liquefaction section 106, fermentation section 108, or ethanol production section 110. For example, the fermentation time may be estimated using rate tags, such as flour feed rate, beer feed rate, slurry to fermentation rate, etc. The fermentation solids may be estimated using solids measurements from samples in a lab, slurry density measurements, flour mass, etc. The temperature may be determined from temperature readings in the liquefaction section 106, temperature readings the fermentation section 108, ambient temperature readings, and/or temperature differentials in a cooling plant of the fermentation section 108. The fermentation products and fermentation by-products may be estimated using measurements from samples in a lab, such as high-performance liquid chromatography (HPLC), or in-line measurement devices, such as spectrometers (e.g., FTIR spectrometers, MIR spectrometers, and NIR spectrometers).

The yeast blend ratio may be determined based on two or more types of yeast exhibiting at least one unique fermentation characteristic, including but not limited to ability to produce different fermentation products or co-products (including but not limited to lipids, alcohols, proteins, esters, oils, etc.) substrate conversion efficiency (where substrate is C5 or C6 sugars), low by-product formation (glycerol, acetic acid, lactic acid, succinic acid, carbon dioxide, and biomass), robustness (tolerance to one or more of high-temperature and/or high concentrations of organic acids, fusel alcohols, osmotic stress), fermentation kinetics (fast or slow fermenting at the beginning, middle, or end of fermentation), nitrogen utilization efficiency (ability to use wide variety of amino nitrogen sources and/or decreased exogenous sources of urea (urea and/or ammonia)), fermentation time (less than 48, 48 to 54, 54 to 65, or above 65 hours). For example, a yeast blend may include a yeast with high substrate conversion efficiency (Innova Element) paired with a yeast developed for robustness (Innova Force). Non-limiting examples of additional yeast having one or more of the above unique fermentation characteristics include Innova Force, Innova Drive, Innova Element, Innova Fit, Tranform Yield, YP3, CV5, Synerxia, Ruby, Sapphire, Ethanol Red.

The yeast blend may include yeast types capable of metabolizing the same sugars. For example, the high-conversion efficiency yeast and robustness yeast may both be capable of metabolizing C5 sugars. The yeast blend may include yeast types capable of metabolizing different sugars. For example, the high-conversion efficiency yeast capable of metabolizing C5 sugars and the robustness yeast may be capable of metabolizing C6 sugars. Various yeast combinations (e.g., high-conversion efficiency and robustness or C5 and C6 yeasts) are within the scope of this disclosure.

The input scheme may include a feed rate and/or a feed scheme, e.g. of grain. The one or more controllers may control a feeder input device according to the feed rate/feed scheme. The feed scheme may include a first feed rate or first feed amount for a first feeder input device, e.g. for feeding grain or other raw material to the preparation section 104 of the bioethanol system 102. The one or more controllers may control a first feeder input device, such as a rotary valve, according to the feed rate/feed scheme, such as a first feed rate or first feed amount (e.g., a first feed control parameter FCP_1). The feed scheme may further include additional feed control parameters, such as a second feed control parameter (FCP_2), a third feed control parameter (FCP_3), etc., configured to define the control parameters of respective feeder input devices, such as a second feeder input device and a third feeder input device.

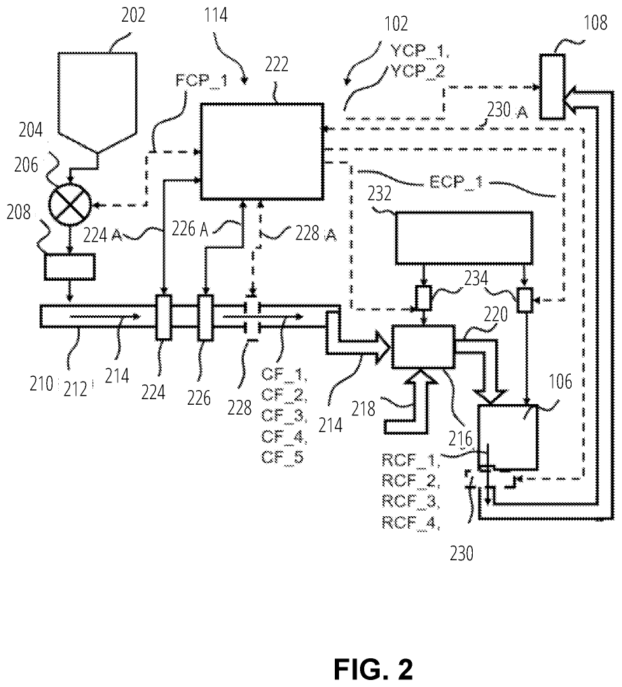

FIG. 2 shows a more detailed block diagram of the bioethanol system 102 according to the present disclosure. The preparation section 104 may include a grain container or grain bin 202 having an output connected to feeder input device 204 including a rotary feeder 206. The feeder input device 204 feeds grain, such as corn, wheat, barley, rye, milo, sago, cassava, tapioca, sorghum, oat, rice, peas, beans, beats, sweet potatoes, or mixtures thereof, to a milling device 208 that may include one or more hammer mills and/or grinders. The milling device outputs grain flour to conveyor 210, such as a conveyor belt 212. The conveyor 210 feeds the grain flour 214 to a mixing device 216, where the grain flour 214 is mixed with liquid 218, such as backset cook water and/or fresh water. The output or slurry 220 from the mixing device 216 may then be fed to liquefaction section 106.

The control system 114 includes a controller 222 including one or more processors and an interface. The control system 114 may include one or more sensors wired or wirelessly connected to the interface of the controller 222. The one or more sensors may include a first sensor 224 and/or a second sensor 226 respectively feeding first sensor data 224A and second sensor data 226A to the controller 222. The one or more sensors may further include a third sensor 228 feeding third sensor data 228A to the controller 222. The one or more sensors may also include a fourth sensor 230 feeding fourth sensor data 230A to the controller 222.

The first sensor 224 is arranged at the conveyor 210 and configured to sense or measure one or more properties of the grain flour 214 output from the milling device 208. The first sensor data 224A may include one or more properties, such as a weight, of the grain flour 214 passing the first sensor 224, a speed of the grain flour 214 passing the first sensor 224, and/or spectrometer data of the grain flour 214 passing the first sensor 224.

The second sensor 226 and the third sensor 228 may also be arranged at the conveyor 210 and configured to sense or measure one or more properties of the grain flour 214, such as a weight of the grain flour 214 passing the second sensor 226 and/or the third sensor 228, a speed of the grain flour 214 passing the second sensor 226 and/or the third sensor 228, and/or spectrometer data of the grain flour 214 passing the second sensor 226 and/or the third sensor 228.

The one or more processors of the controller 222 are configured to obtain a grain flour flow of grain flour 214 and/or component flows of (CF_1, CF_2, CF_3, CF_4, CF_5, etc.) by receiving first sensor data 224A, second sensor data 226A, and/or third sensor data 228A.

In some embodiments, the fourth sensor 230 may be a spectrometer including one or more NIR, FTIR, or MIR spectrometers for measuring spectrometer data of a liquefact/liquefied mash as the fourth sensor data 230A. The fourth sensor 230 may be situated in the outlet of the liquefaction tank of liquefaction section 106 to obtain the spectrometer data of the liquefact/liquefied mash as the fourth sensor data 230A. The controller 222 may determine remaining component flows RCF_1, RCF_2, etc., such as a remaining starch flow, a remaining protein flow, a remaining fiber flow, a remaining fat flow, etc., by comparing component flow CF_x from one of the earlier sensors 224, 226, 228 to the associated component flow measured by the fourth sensor 230. For example, the remaining first component flow RCF_1 or remaining first component flow amount may be determined as a fraction of the first component flow CF_1 remaining at the end of liquefaction determined based on the difference of the first component flow or first component amount determined by the earlier sensor data 224A, 226A, or 228A and the first component flow or first component amount of the fourth sensor data 230A.

In some embodiments, the fourth sensor 230 may be configured to determine properties of the liquefied mash 220 exiting the liquefaction section 106 and/or entering the fermentation section 108. For example, the fourth sensor 230 may include one or more of a velocity sensor or flow rate sensor to measure a flow rate of the liquefied mash, a density meter or weight meter to measure a liquefied mash density, a temperature sensor to measure a temperature of the liquefied mash 220, or a spectrometer to determine the organic compounds contained in the liquefied mash 220.

The one or more processors of the controller 222 are configured to determine an input scheme based on the grain flour flow GFF and/or one or more of the sensor readings from the sensors 2224, 226, 228, 230. The input scheme includes one or more of an enzyme dosing scheme, a yeast dosing scheme and feed rate/feed scheme.

For example, the fourth sensor 230 may be a spectrometer including one or more NIR or MIR sensors for measuring spectrometer data of a liquefact/liquefied mash as the fourth sensor data 230A. The fourth sensor 230 may be situated in the outlet of the liquefaction tank of liquefaction section 106 to obtain the spectrometer data of the liquefact/liquefied mash as the fourth sensor data 230A. To obtain a grain flour flow of grain flour 214 optionally comprises to determine a remaining first component flow RCF_1, such as a remaining starch flow, based on the spectrometer data from one of the other sensors 224, 226, 228, the fourth sensor data/spectrometer data 230A, and the grain flour flow GFF. For example, the remaining first component flow RCF_1 or remaining first component flow amount may be determined as a fraction of the first component flow CF_1 remaining at the end of liquefaction determined based on the difference of the first component flow or first component amount determined by the spectrometer sensor data and the first component flow or first component amount of the fourth sensor data 230A. The remaining first component flow or first component amount at the end of liquefaction may be used to adjust dosing of the first enzyme or first enzyme flow rate. For example, if the first enzyme is alpha-amylase and the first component flow is starch, the fourth sensor/sensor device can detect sugar profiles (e.g., DP1, DP2, DP3, DP4, etc.) that can be used by the control system to determine whether adjustments in alpha-amylase dosing need to be made based on the amount of starch remaining post-liquefaction as detected by the fourth sensor or fourth sensor device relative to the amount of starch present in the grain flour flow measured by the third sensor or third sensor device.

To obtain a grain flour flow of grain flour 214 optionally comprises to determine a remaining second component flow RCF_2, such as a remaining protein flow, based on pectrometer data, the fourth sensor data/spectrometer data 230A, and the grain flour flow GFF. For example, the remaining second component flow RCF_2 or remaining second component flow amount may be determined as a fraction of the second component flow CF_2 remaining at the end of liquefaction determined based on the difference of the second component flow or second component amount determined by the spectrometer sensor data and the second component flow or second component amount of the fourth sensor data 230A. The remaining second component flow or second component amount at the end of liquefaction may be used to adjust dosing of the second enzyme or second enzyme flow rate. For example, if the second enzyme is protease and the second component flow is protein, the fourth sensor/sensor device can detect protein (e.g., soluble protein) that can be used by the control system to determine whether adjustments in protease dosing need to be made based on the amount of protein remaining post-liquefaction as detected by the fourth sensor or fourth sensor device relative to the amount of protein present in the grain flour flow measured by the third sensor or third sensor device.

To obtain a grain flour flow of grain flour 214 optionally includes to determine a remaining third component flow RCF_3, such as a remaining fiber flow, based on the spectrometer data, the fourth sensor data/spectrometer data 230A, and the grain flour flow GFF. For example, the remaining third component flow RCF_3 or remaining third component flow amount may be determined as a fraction of the third component flow CF_3 remaining at the end of liquefaction determined based on the difference of the third component flow or third component amount determined by the spectrometer sensor data and the third component flow or third component amount of the fourth sensor data 230A. The remaining third component flow or third component amount at the end of liquefaction may be used to adjust dosing of the third enzyme or third enzyme flow rate. For example, if the third enzyme is xylanase and the third component flow is hemicellulose, the fourth sensor/sensor device can detect hemicellulose, or the xylose and/or arabinose hydrolysis products of xylanase on the hemicellulose, that can be used by the control system to determine whether adjustments in protease dosing need to be made based on the amount of hemicellulose or xylose and/or arabinose hydrolysis products of xylanase on the hemicellulose, remaining post-liquefaction as detected by the fourth sensor or fourth sensor device relative to the amount of hemicellulose present in the grain flour flow measured by the third sensor or third sensor device.

The one or more processors of the controller 222 are configured to determine an input scheme based on the grain flour flow GFF and/or one or more component flows, such as first component flow CF_1 of a first component of the grain flour and/or a second component flow CF_2 of a second component of the grain flour and/or a third component flow CF_3 of a third component of the grain flour and/or a fourth component flow CF_4 of a fourth component of the grain flour and/or a fifth component flow CF_5 of a fifth component of the grain flour. The input scheme includes one or more of an enzyme dosing scheme, a yeast dosing scheme and feed rate/feed scheme, respectively defining enzyme dosing, yeast dosing, and grain feed in the bioethanol system.

The one or more processors of the controller 222 are configured to control one or more input devices of the bioethanol system according to the input scheme. For example, the one or more processors of the controller 222 may be configured to transmit feed control scheme/feed control parameter(s) FCP to the input feeder input device 204. The feeder input device 204 operates accordingly to feed grain from grain bin 202 to milling device 208.

The preparation section 104 and/or the liquefaction section 106 may comprise an enzyme system comprising one or more enzyme containers including first enzyme container 232 and one or more enzyme input devices including a first enzyme input device 234. The one or more processors of the controller 222 may be configured to transmit enzyme dosing scheme/one or more enzyme control parameters including ECP_1 to the first enzyme input device 234 configured to input first enzyme in the first enzyme container 232 to the mixing device 216, thereby adding and mixing first enzyme to the grain flour 214 and liquid 218 according to the enzyme dosing scheme.

The one or more processors of the controller 222 may be configured to determine a yeast dosing scheme, the yeast dosing scheme comprising a first yeast flowrate of a first yeast. The controller 222 may control the input devices comprises according to the yeast dosing scheme by transmitting a first yeast control parameter YCP_1 and/or a second yeast control parameter YCP_2 to a yeast system 300.

FIG. 3 illustrates the yeast system 300. The yeast system 300 may include a first yeast container 304 and a second yeast container 306. The yeast containers 304 and 306 may be coupled to a mixing chamber 302 through respective yeast input devices 310, 312. The mixing chamber 302 may be a part of the fermentation section 108 where the yeast from the yeast system 300 mixes with the liquefied mash 220 to begin the fermentation process. In some embodiments, the mixing chamber 302 may be separate from the fermentation section 108, such that a yeast blend is injected into the fermentation section 108 from the mixing chamber 302.

As described above, the yeast input devices 310, 312 may be controlled based on the yeast dosing scheme. The yeast dosing scheme may include both a total amount or total flow rate of yeast and a yeast blend ratio, which may determine the ratio of a first yeast from the first yeast container 304 to be input through the first yeast input device 310 and a second yeast from the second yeast container 306 to be input through the second yeast input device 312.

In some embodiments, one or more of the first yeast and the second yeast may be a dry yeast. The dry yeast may be re-hydrated in a rehydration chamber 308 before being injected through the respective yeast input device 312, 310. For example, as illustrated in FIG. 3, if the second yeast is a dry yeast, the dry yeast may pass into the rehydration chamber 308 after leaving the second yeast container 306 and before reaching the second yeast input device 312. In the rehydration chamber 308 the dry second yeast may be rehydrated into a cream yeast. Once the dry second yeast is rehydrated, the second yeast may pass through the second yeast input device 312 as a cream yeast, which may be more precisely controlled than the dry yeast.

The yeast system 300 may be maintained at a temperature lower than room temperature (e.g., less than about 20° C.). For example, the yeast system 300 may be refrigerated. Maintaining the yeast system 300 at a temperature below room temperature may maintain the yeast in a substantially stable condition. Maintaining the yeast in a substantially stable condition may allow the yeast to reserve the energy to be released during the fermentation process in the fermentation section 108 rather than releasing the energy in the yeast system 300 prior to being inserted into the fermentation section 108.

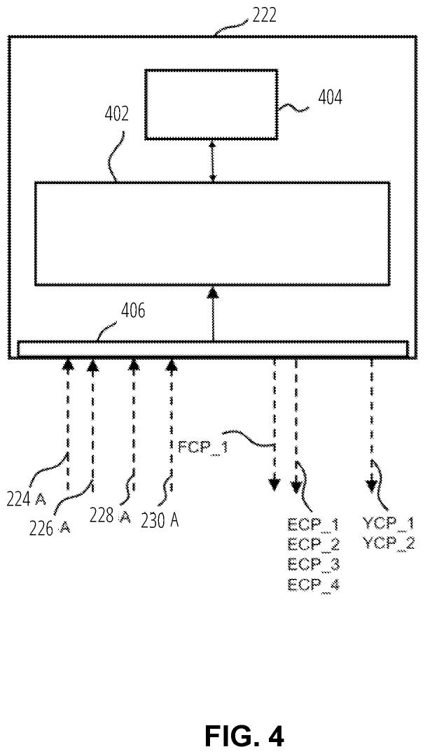

FIG. 4 shows a controller 222 of a control system 114 according to the present disclosure. The controller 222 may include one or more processors 402, memory 404 and an interface 406. The interface 406 connects (wired and/or wirelessly) the controller 222 to sensor(s), such as sensors 224, 226, 228, 230 and/or input device(s), such as input devices 204, 234, of the bioethanol system 102. The one or more processors 402 are connected to memory 404, the memory 404 storing input scheme and/or system configuration parameters or other settings relevant for the operation of controller 222.

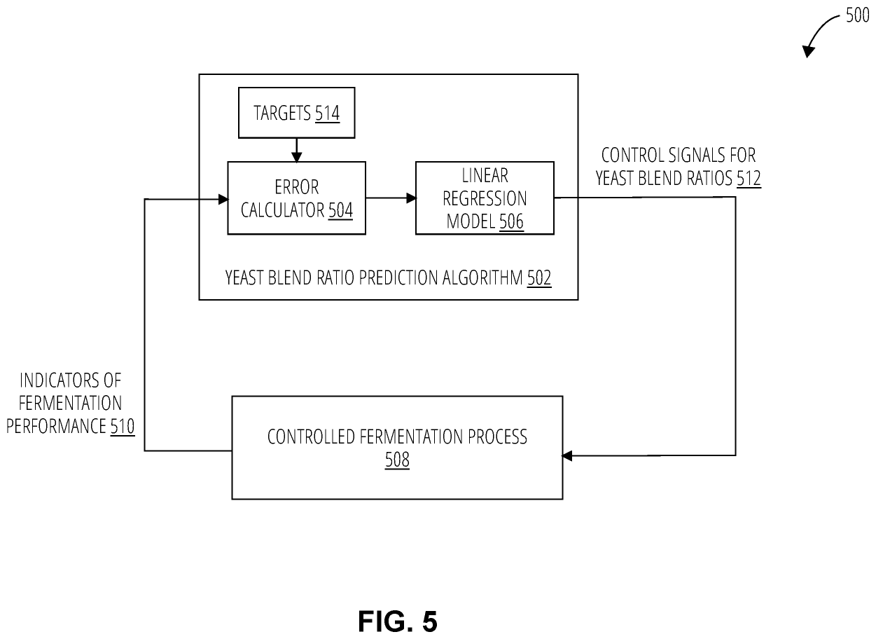

FIG. 5 illustrates an example control system 500, according to various embodiments of the present disclosure. For example only, control system 500 may include or may be part of control system 114 of FIG. 1. System 500 includes a yeast blend ratio prediction algorithm 502, which includes an error calculation unit 504 and a model 506 (e.g., a linear regression model). System 500 further includes a controlled fermentation process 508 (e.g., performed by a system such as system 102). As will be appreciated, system 500 may include one or more processors (e.g., processors 402 of FIG. 4) for carrying out various embodiments disclosed herein.

Error calculator 504 may be configured to determine an error between target data (e.g., benchmark experimental data and/or target process conditions) 514 and indicators of fermentation performance 510. For example, indicators of fermentation performance 510, which may include various indicators and/or stressors (also referred to herein as “process data”) associated with the controlled fermentation process 508. For example only, the indicators and/or stressors may include fermentation time (e.g., estimated using plant rate tags such as beer feed rate, slurry to fermentation rate, etc.), fermentation solids (e.g., estimated using solids measurements from the lab, mash density, Ronin flour mass, etc.), temperature (e.g., estimated using temperature tags from slurry, liquefaction, etc. and/or based on a temperature differential from a cooling plant that would integrate the ambient temperature outside of the plant with the ability of the facility to cool the fermentation via heat exchangers), and fermentation product and/or fermentation by-product content, such as ethanol, glycerol and organic acids (e.g., acetic acid, lactic acid, etc.) (e.g., estimated using HPLC measurements from the lab and/or or in-line measurements from one or more instruments).

Responsive to receipt of an error signal from error calculator 504, linear regression model 506 may determine, based on the error signal, a yeast blend ratio. Further, linear regression model 506 may generate one or more control signals that may be received by the controlled fermentation process 508 for controlling one or more yeast dosing skids based on the determined yeast blend ratio. Linear regression model 506 may be a least-squares regression model.

Yeast blend ratio prediction algorithm 502 may determine control signals 512 for yeast blend ratio determined by model 506. Control signals 512 may be configured to directly control actuators at controlled fermentation process 508 or may be indicative of control actions, for example only, including values for metering-in volumes of respective yeasts of a yeast blend, values for boluses of volumes of respective yeasts of a yeast blend, or values for ratios that may be utilized by a dosing control mechanism.

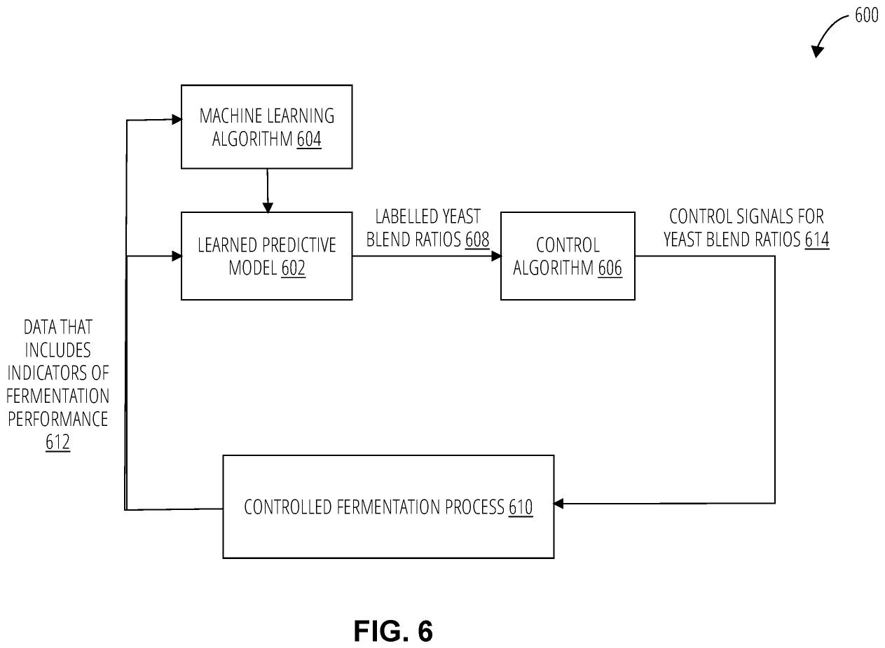

FIG. 6 illustrates an example control system 600, according to various embodiment of the present disclosure. For example only, control system 600 may include or may be part of control system 114 of FIG. 1. System 600 includes a learned predictive model 602, a machine learning algorithm 604, a control algorithm 606, and a controlled fermentation process 610 (e.g., performed by a system such as system 102). As will be appreciated, system 600 may include one or more processors (e.g., processors 402 of FIG. 4) for carrying out various embodiments disclosed herein.

The learned predictive model 602 and the machine learning algorithm 604 may be configured to receive data 612, which may include indicators of fermentation performance of controlled fermentation process 610. Such indicators of fermentation performance may be known, unknown, or a combination thereof. Similar to as described above with regard to control system 500, data 612 may include various indicators and/or stressors (also referred to herein as “process data”) associated with the controlled fermentation process 610. For example only, the indicators and/or stressors associated with the controlled fermentation process 610 may include fermentation time, fermentation solids, temperature, and fermentation products (e.g., ethanol) or fermentation by-products, such as glycerol and organic acid (e.g. acetic acid) content. Feature vectors of indicators may be continuously identified and utilized by control system 600.

Based on data 612 and/or data received from a machine learning algorithm 604, the learned predictive model 602 may label various yeast blend ratio (respectively “labelled yeast blend ratios 608”) that include information that identifies a predicted yield for a given yeast blend ratio. Control algorithm 606 may utilize the labelled yeast blend ratios 608 to determine a specific yeast blend ratio and generate one or more control signals 614 that may be received by the controlled fermentation process 610 for controlling one or more yeast dosing skids based on the determined yeast blend ratio.

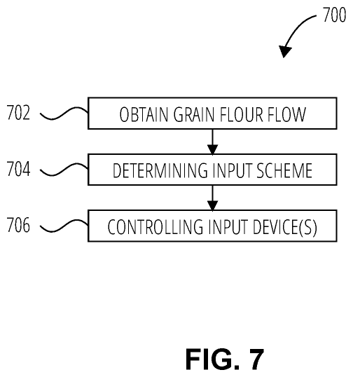

FIG. 7 shows a flowchart of an exemplary method 700 of producing bioethanol in a bioethanol system, the method 700 including obtaining a grain flour flow of grain flour in act 702, determining an input scheme based on the grain flour flow in act 704, and controlling one or more input devices of the bioethanol system according to the input scheme in act 706.

The embodiments of the disclosure described above and illustrated in the accompanying drawing figures do not limit the scope of the invention, since these embodiments are merely examples of embodiments of the invention, which is defined by the appended claims and their legal equivalents. Any equivalent embodiments are intended to be within the scope of this disclosure. Indeed, various modifications of the present disclosure, in addition to those shown and described herein, such as alternative useful combinations of the elements described, may become apparent to those skilled in the art from the description. Such modifications and embodiments are also intended to fall within the scope of the appended claims and their legal equivalents.

The invention is further defined in the following paragraphs:

1. A control system for a bioethanol system, the control system comprising a controller comprising one or more processors and an interface, wherein the one or more processors are configured to:
obtain a grain flour flow of grain flour;
determine an input scheme based on the grain flour flow; and
control one or more input devices of the bioethanol system according to the input scheme.

2. Control system according to paragraph 1, wherein to determine an input scheme comprises to determine an enzyme dosing scheme, the enzyme dosing scheme comprising a first enzyme flowrate for a first enzyme, and wherein to control one or more input devices comprises to control a first enzyme input device according to the enzyme dosing scheme.

3. Control system according to paragraph 2, wherein to control an input device comprises to control the first enzyme input device according to the first enzyme flowrate.

4. Control system according to any of paragraphs 2-3, wherein the enzyme dosing scheme comprises a second enzyme flowrate for a second enzyme, and wherein to control an input device comprises to control a second enzyme input device according to the second enzyme flowrate.

5. Control system according to any of paragraphs 2-4, wherein the first enzyme and second enzyme are selected from alpha-amylase, endoglucanase, glucoamylase, phospholipase, phytase, protease, pullulanase, and xylanase or is a first enzyme composition comprising one or more of alpha-amylase, endoglucanase, glucoamylase, phospholipase, phytase, protease, pullulanase, and xylanase

6. Control system according to any of paragraphs 1-5, wherein to determine an input scheme comprises to determine a yeast dosing scheme, the yeast dosing scheme comprising a first yeast flowrate of a first yeast, and wherein to control one or more input devices comprises to control a first yeast input device according to the yeast dosing scheme.

7. Control system according to paragraph 6, wherein to control an input device comprises to control the first yeast input device according to the first yeast flowrate.

8. Control system according to any of paragraphs 6-7, wherein the yeast dosing scheme comprises a second yeast flowrate of a second yeast, and wherein to control an input device comprises to control a second yeast input device according to the second yeast flowrate.

9. Control system according to any of paragraphs 6-8, wherein the first yeast and/or the second yeast is selected from Saccharomyces, Rhodotorula, Schizosaccharomyces, Kluyveromyces, Pichia, Hansenula, Rhodosporidium, Candida, Yarrowia, Lipomyces, Cryptococcus,and Dekkera sp., or is a first yeast composition comprising one or more of Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp.

10. Control system according to any of paragraphs 1-9, wherein to determine an input scheme comprises to determine a feed rate, and wherein to control one or more input devices comprises to control a feeder input device according to the feed rate.

11. Control system according to any of paragraphs 1-10, wherein the control system comprises a weight meter for provision of weight data of grain flour and wherein to obtain a grain flour flow comprises to determine the grain flour flow based on the weight data.

12. Control system according to any of paragraphs 1-11, wherein the control system comprises a spectrometer for provision of spectrometer data of grain flour and wherein to obtain a grain flour flow comprises to determine a first component flow of a first component of the grain flour based on the spectrometer data and the grain flour flow.

13. Control system according to paragraph 12, wherein the first component is one of a starch, a protein, a fiber, a fat, and a moisture content.

14. Control system according to any of paragraphs 12 or 13, wherein to determine a first enzyme flowrate for a first enzyme is based on the first component flow.

15. Control system according to any of paragraphs 12-14, wherein to obtain a grain flour flow comprises to determine a second component flow of a second component of the grain flour based on the spectrometer data and the grain flour flow.

16. Control system according to paragraph 15, wherein the second component is one of a starch, a protein, a fiber, a fat, and a moisture content.

17. Control system according to any of paragraphs 15 or 16, wherein to determine a second enzyme flowrate for a second enzyme is based on the second component flow.

18. Control system according to any of paragraphs 1-17, wherein grain flour is obtained from a single feedstock.

19. Control system according to any of paragraphs 1-18, wherein grain flour is obtained from multiple feedstocks, such as at least two, at least three, at least 4, at least 5 feedstocks.

20. Control system according to any of paragraphs 1-19, wherein grain flour is obtained from feedstocks selected from the group comprising corn, wheat, barley, rye, milo, sago, cassava, tapioca, sorghum, oat, rice, peas, beans, beats, sweet potatoes, other sources of starch, such as e.g., starch waste by-products, or mixtures thereof.

21. Method of producing bioethanol in a bioethanol system, the method comprising:
obtaining a grain flour flow of grain flour;
determining an input scheme based on the grain flour flow; and
controlling one or more input devices of the bioethanol system according to the input scheme.

22. Method or system according to any of the preceding paragraphs, wherein the control system is configured to optimize ethanol yield by simultaneously and continuously:
determining a feed rate and controlling one or more input devices to control a feeder input device according to the feed rate;
determining an enzyme dosing scheme and to control one or more input devices according to the enzyme dosing scheme; and optionally
determining a yeast blend ratio based on the one or more process conditions, and transmit a first yeast control parameter to the first yeast input device based on the yeast blend ratio, and transmit a second yeast control parameter to the second yeast input device based on the yeast blend ratio.

23. Method or system according to any of the preceding paragraphs, wherein the enzyme dosing scheme and/or the yeast blend ratio is based on a determination of the grain flour flow, and/or the first component flow and/or the second component flow.

24. The method or system according to any of the preceding paragraphs, wherein the ethanol yield is increased compared to an equivalent method where no controlling system is applied.

25. The method or system according to any of the preceding paragraphs, wherein the ethanol yield is maintained, and enzyme dosing is decreased, compared to an equivalent method where no controlling system is applied.

26. The method or system according to any of the preceding paragraphs, wherein the variability of enzyme dosing is reduced compared to an equivalent method where no controlling system is applied.

27. The method or system according to any of the preceding paragraphs, wherein the variability of the grain flour input into the bioethanol system is reduced compared to an equivalent method where no controlling system is applied.

28. The method or system according to any of the preceding paragraphs, wherein the feedstock is a mixture of at least corn and milo or at least corn and wheat.

29. A yeast injection system comprising:
a first yeast container coupled to a mixing chamber through a first yeast input device;
a second yeast container coupled to the mixing chamber through a second yeast input device; and
a controller configured to measure one or more process conditions associated with the mixing chamber and determine a yeast blend ratio based on the one or more process conditions, wherein the controller is configured to control the first yeast input device and the second yeast input device based on the yeast blend ratio.

30. The yeast injection system of paragraph 29, wherein the yeast injection system is maintained at a temperature below room temperature.

31. The yeast injection system of paragraph 29, wherein the one or more process conditions are selected from the group consisting of a fermentation time, a fermentation solid content, a temperature, an organic acid content.

32. The yeast injection system of paragraph 29, wherein the mixing chamber comprises a fermentation section of a bioethanol production system.

33. The yeast injection system of paragraph 29, further comprising a rehydration chamber positioned between the second yeast container and the second yeast input device, the rehydration chamber configured to rehydrate a dry yeast.

34. A bioethanol production system comprising:
a fermentation section;
a controller configured to measure one or more process conditions associated with the bioethanol production system; and
a yeast injection system coupled to the fermentation section, the yeast injection system comprising:
a first yeast container coupled to the fermentation section through a first yeast input device;
a second yeast container coupled to the fermentation section through a second yeast input device; and
wherein the controller is configured to determine a yeast blend ratio based on the one or more process conditions, and transmit a first yeast control parameter to the first yeast input device based on the yeast blend ratio, and transmit a second yeast control parameter to the second yeast input device based on the yeast blend ratio.

35. The bioethanol production system of paragraph 34, wherein the one or more process conditions are selected from the group consisting of a fermentation time, a fermentation solid content, a temperature, an organic acid content.

36. The bioethanol production system of paragraph 34, the yeast injection system further comprising a rehydration chamber positioned between the second yeast container and the second yeast input device, the rehydration chamber configured to rehydrate a dry yeast.

37. A method of producing bioethanol comprising:
forming a slurry from a mixture of a grain flour, water;
liquefying starch in the slurry using one or more enzymes to form a mash;
measuring one or more properties of the mash;
injecting a blend of a first yeast and a second yeast into the mash, wherein a ratio of the first yeast to the second yeast is determined based on the one or more properties of the mash;
controlling the ratio of the first yeast and the second yeast through a first yeast input device and a second yeast input device;
fermenting fermentable sugars in the mash with the blend of the first yeast and the second yeast; and
extracting bioethanol from the fermented mash.

38. A control system, comprising:
one or more processors configured to communicatively couple with a bioethanol system, the one or more processors further configured to:
sense one or more indicators associated with the fermentation system;
determine a yeast blend ratio based at least partially on the one or more indicators;
generate one or more control signals based on the determined yeast blend; and
convey the one or more control signals to the fermentation system.

39. The control system of paragraph 38, further comprising:
an error calculator including at least one processor of the one or more processors and configured to generate an error signal based on the one or more indicators and target data; and
a model including at least one processor of the one or more processors and configured to determine the yeast blend ratio based on the error signal.

40. The control system of paragraph 39, wherein the model includes a linear regression model.

41. The control system of paragraph 38, wherein the one or more indicators are selected from the group consisting of a fermentation time, a fermentation solid content, a temperature, an organic acid content.

42. A control system, comprising:
one or more processors configured to communicatively couple with a fermentation system, the one or more processors further configured to:
receive data including one or more indicators associated with the fermentation system;
determine one or more yeast blend ratios based at least partially on the one or more indicators;
generate one or more control signals based on the determined yeast blend; and
convey the one or more control signals to the fermentation system.

43. The control system of paragraph 42, further comprising:
a learned predictive model including at least one processor of the one or more processors and configured to determine the one or more yeast blend ratios based at least partially on the data; and
a control block configured to generate the one or more control signals based on the determined yeast blend.

44. The control system of paragraph 42, wherein the one or more indicators are selected from the group consisting of a fermentation time, a fermentation solid content, a temperature, an organic acid content.

45. A method, comprising:
sensing one or more indicators associated with a fermentation system;
determining a yeast blend ratio based at least partially on the one or more indicators;
generating one or more control signals based on the determined yeast blend; and
conveying one or more control signals to the fermentation system.

46. The method of paragraph 45, further comprising determining an error between the one or more indicators and target data, wherein determining the yeast blend ratio comprising determining the yeast blend ratio based at least partially on the determined error.

47. A method, comprising:
receiving data including more indicators associated with a fermentation system;
determining one or more yeast blend ratios based at least partially on the data;
generating one or more control signals based on the determined one or more yeast blends; and
conveying one or more control signals to the fermentation system.

48. The method of paragraph 47, wherein receiving the data comprises receiving the data at a learned predictive model configured to determine the one or more yeast blend ratios.

### EXAMPLES

### Example 1

Ethanol producers have failed to properly optimize their operations due to high process variability in the conventional dry grind ethanol production process, chiefly caused by the inability to measure their grain and thus to dose enzymes appropriately. The inability to measure grain and dose enzyme means that the mash entering fermentation is constantly changing, but those changes are largely invisible to operators. Any attempt to standardize or optimize fermentation is bound to be unsuccessful as the attempt is made against a largely invisible, moving target.

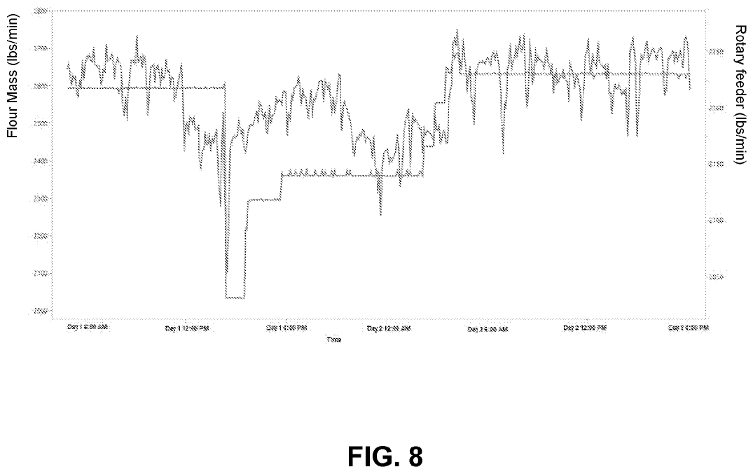

Typically, ethanol producers add enzymes into the process at a constant rate and then attempt to grind a constant amount of grain with the goal of generating a constant enzyme dose (on a weight of enzyme/weight of corn basis). The main error occurs because grain grind is controlled using the rotary feeders of the mills, which are highly inaccurate as shown in FIG. 8.

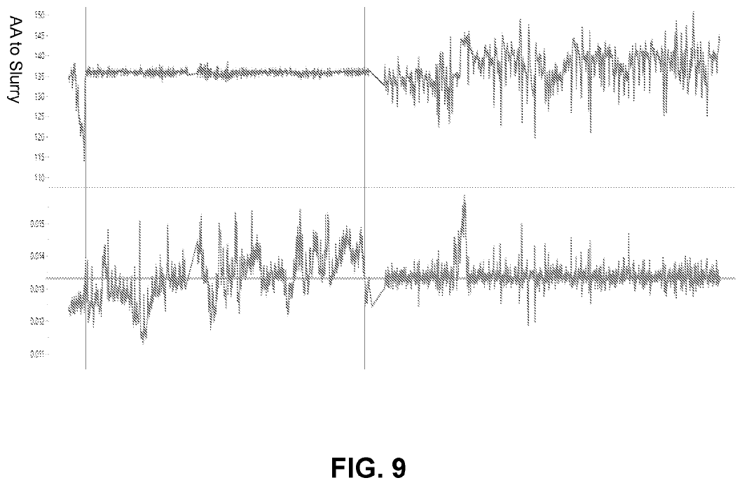

Referring to FIG. 8, when the red line shown is flat, the ethanol producers think that they are grinding a constant amount of grain, but the mass of ground flour (shown in the blue line) is actually highly variable. If the ethanol producer is pumping enzyme at a constant rate, but the grain mass is highly variable, then the resulting enzyme dose will be highly variable, as shown in the left side of FIG. 9.

The bioethanol control system of the present disclosure accurately measures flour mass and solves the above problem in two important ways. First, control logic reduces variability in grind by adjusting the rotary feeders in real time so that ethanol plant only grinds as much flour is needed to optimize their process according to predetermined targets. Second, control logic modulates the enzyme rate based on the actual amount of flour substrate so that the correct amount of enzyme is added for the amount of flour that has been ground, resulting in a stable weight/weight dose of enzyme over time. This effect is shown in the right side of FIG. 9, where the alpha-amylase (AA) rate is allowed to change with flour mass (top) resulting in a dose with much lower variability (bottom). Ultimately, this massive reduction in variability of grain flour and enzyme dose means that the mash entering each fermenter is as constant in quality as it can be. This translates into much lower variability in fermentation outcomes, which makes production more stable and predictable.

### Example 2

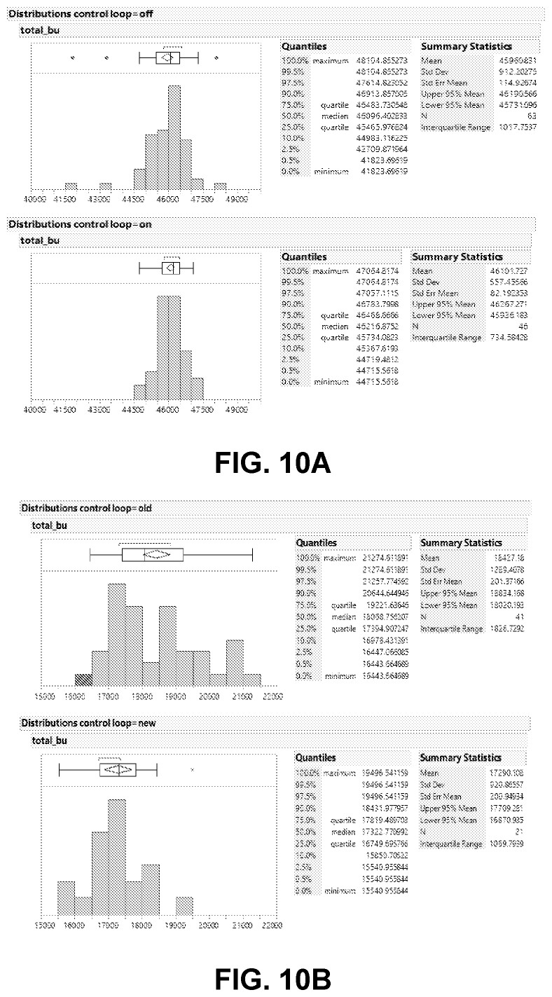

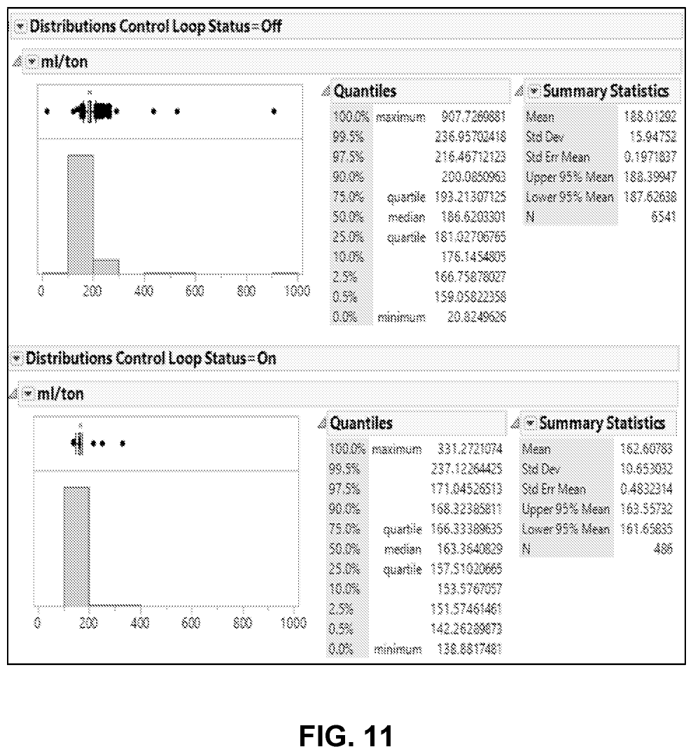

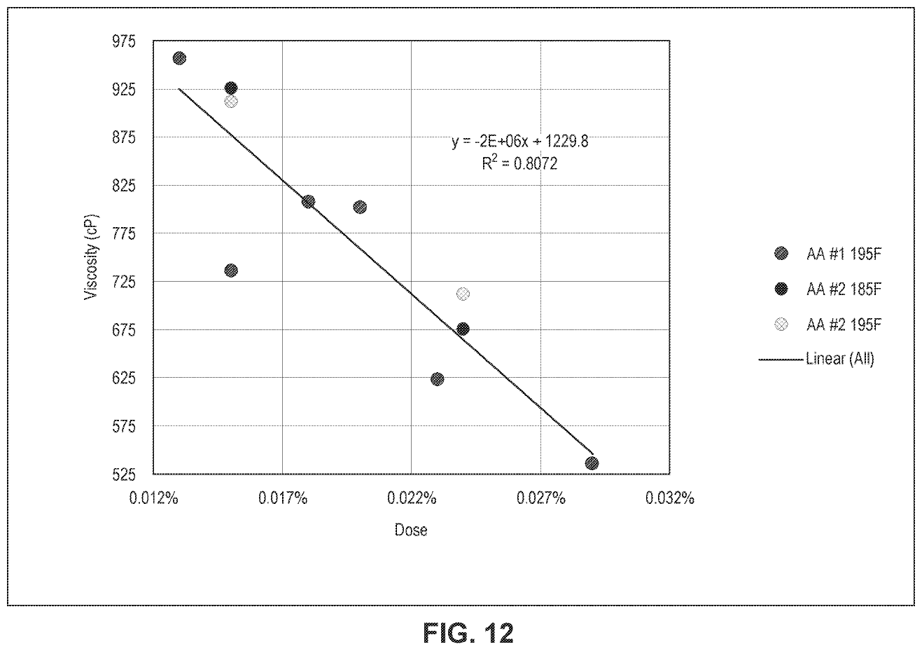

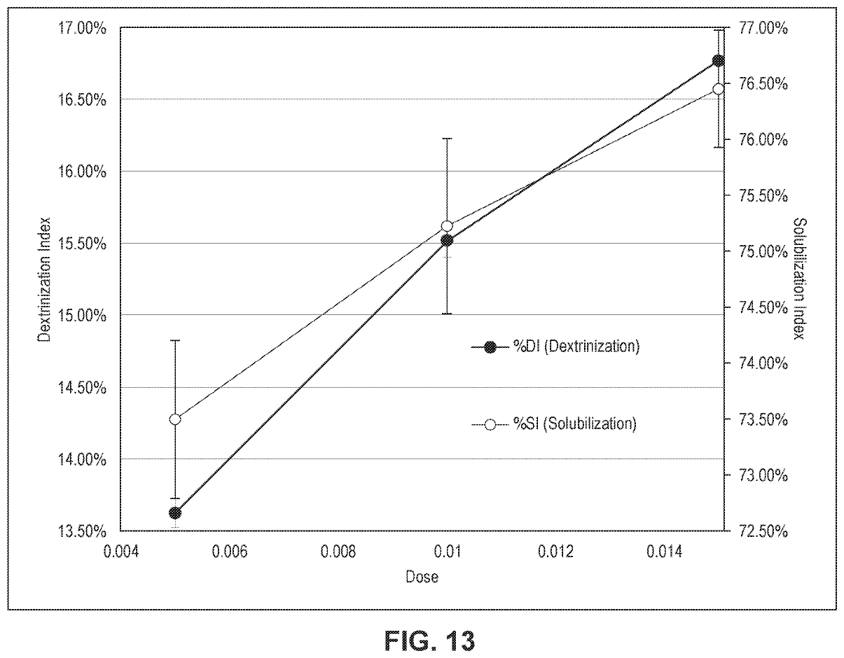

The control system of the present disclosure enables process control that significantly reduces observed variability in both the grinding of corn (See FIG. 10A & FIG. 10B) and enzyme dosing (See FIG. 11) in the dry-grind ethanol process. This is very important as process variability is a critical factor that negatively impacts the performance and profitability of an ethanol plant, and high variability makes it very challenging for ethanol plants to achieve the statistical power required to prove the value of process improvements. If an ethanol plant has high variation around their target alpha amylase dose, this means that they are overdosing and underdosing a significant amount of the time. Overdosing is a waste of alpha amylase and is costly for ethanol plants. Underdosing results in substantial increases in mash viscosity (See FIG. 12). Increased viscosity mash is harder to pump and requires increased, costly energy consumption, and very high levels of viscosity can result in fouling that can shut down production altogether. Underdosing of alpha amylase also results in lower dextrinization and solubilization of starch in the liquefaction process (See FIG. 13), which may impact yeast performance in fermentation.

1. A control system for a bioethanol system, the control system comprising a controller comprising one or more processors and an interface, wherein the one or more processors are configured to:
obtain a grain flour flow of grain flour;
determine an input scheme based on the grain flour flow; and
control one or more input devices of the bioethanol system according to the input scheme.

2. Control system according to claim 1, wherein to determine an input scheme comprises to determine an enzyme dosing scheme, the enzyme dosing scheme comprising a first enzyme flowrate for a first enzyme, and wherein to control one or more input devices comprises to control a first enzyme input device according to the enzyme dosing scheme.

3. Control system according to claim 2, wherein to control an input device comprises to control the first enzyme input device according to the first enzyme flowrate.

4. Control system according to claim 3, wherein the enzyme dosing scheme comprises a second enzyme flowrate for a second enzyme, and wherein to control an input device comprises to control a second enzyme input device according to the second enzyme flowrate.

5. Control system according to claim 4, wherein the first enzyme and second enzyme are selected from alpha-amylase, endoglucanase, glucoamylase, phospholipase, phytase, protease, pullulanase, and xylanase or is a first enzyme composition comprising one or more of alpha-amylase, endoglucanase, glucoamylase, phospholipase, phytase, protease, pullulanase, and xylanase

6. Control system according to claim 1, wherein to determine an input scheme comprises to determine a yeast dosing scheme, the yeast dosing scheme comprising a first yeast flowrate of a first yeast, and wherein to control one or more input devices comprises to control a first yeast input device according to the yeast dosing scheme.

7. Control system according to claim 6, wherein to control an input device comprises to control the first yeast input device according to the first yeast flowrate.

8. Control system according to claim 7, wherein the yeast dosing scheme comprises a second yeast flowrate of a second yeast, and wherein to control an input device comprises to control a second yeast input device according to the second yeast flowrate.

9. Control system according to claim 8, wherein the first yeast and/or the second yeast is selected from Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp., or is a first yeast composition comprising one or more of Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp.

10. Control system according to claim 1, wherein to determine an input scheme comprises to determine a feed rate, and wherein to control one or more input devices comprises to control a feeder input device according to the feed rate.

11. Control system according to claim 1, wherein the control system comprises a weight meter for provision of weight data of grain flour and wherein to obtain a grain flour flow comprises to determine the grain flour flow based on the weight data.

12. Control system according to claim 1, wherein the control system comprises a spectrometer for provision of spectrometer data of grain flour and wherein to obtain a grain flour flow comprises to determine a first component flow of a first component of the grain flour based on the spectrometer data and the grain flour flow.

13. Control system according to claim 12, wherein the first component is one of a starch, a protein, a fiber, a fat, and a moisture content.

14. Control system according to claim 12, wherein to determine a first enzyme flowrate for a first enzyme is based on the first component flow.

15. Control system according to claim 12, wherein to obtain a grain flour flow comprises to determine a second component flow of a second component of the grain flour based on the spectrometer data and the grain flour flow.

16. Control system according to claim 12, wherein the control system comprises a spectrometer for provision of spectrometer data of a liquefact/liquefied mash and wherein to obtain a grain flour flow comprises to determine the first component flow of the first component of the grain flour based on the spectrometer data of grain flour and spectrometer data of the liquefact/liquefied mash and the grain flour flow.

17. Method of producing bioethanol in a bioethanol system, the method comprising:
obtaining a grain flour flow or grain flour;
determining an input scheme based on the grain flour flow; and
controlling one or more input devices of the bioethanol system according to the input scheme.

18. A bioethanol production system comprising:
a fermentation section;
a controller configured to measure one or more process conditions associated with the bioethanol production system; and
a yeast injection system coupled to the fermentation section, the yeast injection system comprising a first yeast container coupled to the fermentation section through a first yeast input device;
wherein the controller is configured to determine a yeast dosing scheme comprising a first yeast control parameter indicative of a first yeast flowrate and/or first yeast amount, transmit the first yeast control parameter to the first yeast input device, and to control the first yeast input device according to the yeast dosing scheme.

19. The bioethanol production system of claim 18, further comprising a second yeast container coupled to the fermentation section through a second yeast input device.

20. The bioethanol production system of claim 18, wherein the controller is configured to determine a yeast blend ratio based on the one or more process conditions, and transmit the first yeast control parameter to the first yeast input device based on the yeast blend ratio, and transmit a second yeast control parameter to the second yeast input device based on the yeast blend ratio.
